(19) United States
(12) Patent Application Publication
(10) Pub. No.: US 2010/0145346 A1
(43) Pub. Date: Jun. 10, 2010

(54) TOTAL HIP REPLACEMENT ARTHROPLASTY SUPPORTING JIG AND ITS SYSTEM

(75) Inventors: Yoshio Koga, Niigata (JP); Koichi Kobayashi, Niigata (JP)

Correspondence Address: J. Rodman Steele;Novak Druce & Quigg LLP, 525 Okeechobee Blvd, Suite 1500, West Palm Beach, FL, 33401, US

(73) Assignee: Niigata University, Niigata (JP)

(21) Appl. No.: 11/916,207

(22) PCT Filed: Jun. 15, 2006
(86) PCT No.: PCT/JP2006/312027

§ 371 Date: Nov. 30, 2007

(30) Foreign Application Priority Data
Sep. 6, 2005 (JP) 2005-258217

(51) Int. Cl. A61B 17/56

(52) U.S. Cl. 606/89

(57) ABSTRACT

A jig and a system for supporting artificial hip joint with which an artificial shelf can be arranged at an ideal arranging position more easily at reduced facility cost. Since existing arthroscope (3) and a medical drill generally used in an operating room are fixed to and held on a small jig (4) for supporting artificial hip joint replacement of simple arrangement which can be fixed to the shell of a patient, correct profile of the shell HC of the patient at current moment in time can be recognized easily and surely with an arthroscope (3). Furthermore, since drilling position and direction of the medical drill can be determined using a first two-dimensional coordinates and a second two-dimensional coordinates, the shell HC of the patient can be shaped into ideal profile accurately without using an expensive special device for exclusive use and thereby the artificial shell can be arranged at an ideal position more easily. Since an expensive special device for exclusive use is not required, facility cost can be reduced.

### TECHNICAL FIELD

The present invention relates to a total hip replacement arthroplasty supporting jig and its system, and more particularly to a jig and a system which are suitable for applying to surgery (hereunder, referred to as a total hip replacement arthroplasty) by which a hip joint of a patient is replaced by an artificial hip joint.

### BACKGROUND ART

Recently, the total hip replacement arthroplasty is generally being practiced for a patient under a condition that is difficult to treat by means of leaving a patient's hip joint intact for example, in the patent document 1.

The patent document 1: Japanese patent unexamined application publication No. 9-173365.

And now, with respect to management of the patient's hip joint during surgery using the total hip replacement arthroplasty, a position sensing system is well-known which uses dedicated special equipment equipped with various types of optical systems such as an LED marker or the like.

### DISCLOSURE OF THE INVENTION

### Problems to be Solved by the Invention

In the position sensing system like this, however, the dedicated special equipment is of large size to make it hard to be newly installed in an existing operating room limited in space and besides the dedicated special equipment that is complicated is expensive, leading to high initial equipment cost being incurred. Thus, these forgoing problems have been posed. Furthermore, the dedicated special equipment has had also the problems that each of forms of an acetabulum (part covering a femoral head in a pelvis of the hip joint), femur or the like in the hip joint of a patient could be grasped, whereas the dedicated special equipment could not play a role in determining a locating position of an artificial hip joint during the surgery using the total hip replacement arthroplasty and thus eventually, a skill of a surgeon was required to locate the artificial hip joint at an ideal locating position setting.

Consequently, in view of the forgoing problems, it is an object of the present invention to provide a total hip replacement arthroplasty supporting jig and its system in which an acetabular cup can be even more easily located at an ideal locating position of the patient and the equipment cost can be reduced.

### Means for Solving the Problem

A first aspect of the present invention is a total hip replacement arthroplasty supporting jig equipped with legs which allow a jig main body to be fitted to a patient's acetabulum exposed by dislocating a femoral head during surgery using a total hip replacement arthroplasty, a first locating unit provided in the jig main body to indicate, by a two-dimensional coordinate, an opposite plane substantially opposed to the patient's acetabulum, a second locating unit provided in the jig main body to indicate, by a two-dimensional coordinate, an opposite plane substantially opposed to the patient's acetabulum at a position further distant from the acetabulum than is the first locating unit, an image-taking means holder provided in a freely slidable manner at the first and second locating units to hold an image-taking means for imaging the patient's acetabulum, and a drilling means holder in a freely slidable manner at the first and second locating units to hold a medical drilling means for drilling the patient's acetabulum.

A second aspect of the present invention is a total hip replacement arthroplasty supporting jig in which the image-taking means holder and the drilling means holder are integrated together to hold the image-taking means or the medical drilling means in an mutually exchangeable manner according to need.

A third aspect of the present invention is a total hip replacement arthroplasty supporting system comprising a total hip replacement arthroplasty supporting equipment and its jig. The total hip replacement arthroplasty supporting jig is equipped with legs which allow a jig main body to be fitted to a patient's acetabulum exposed by dislocating a femoral head during surgery using a total hip replacement arthroplasty, a first locating unit provided in the jig main body to indicate, by a two-dimensional coordinate, an opposite plane substantially opposed to the patient's acetabulum, a second locating unit provided in the jig main body to indicate, by a two-dimensional coordinate, an opposite plane substantially opposed to the patient's acetabulum at a position further distant from the patient's acetabulum than the first locating unit, an image-taking means holder provided, in a freely slidable manner, in the first and second locating units to hold an image-taking means for imaging the patient's acetabulum, and a drilling means holder provided, in a freely slidable manner, in the first and second locating units to hold a medical drilling means for drilling the patient's acetabulum. The total hip replacement arthroplasty supporting equipment is equipped with a storage means which stores a postoperative acetabular image data created by converting preliminarily a patient's ideal acetabular form into a three-dimensional image before surgery using the total hip replacement arthroplasty, an image obtaining means which obtains actual image data of the patient's acetabulum imaged by the image-taking means, an acetabular three-dimensional actual image creating means which converts the actual image data into three dimensional image data to creates acetabular three-dimensional actual image data where each of the two-dimensional coordinates in the first and second locating units is associated with each other, and a calculating means which makes the acetabular three-dimensional actual image data and the postoperative acetabular image data retrieved from the storage means coincide with each other to specify a given position of the ideal acetabular form in the postoperative acetabular image data using each of the two-dimensional coordinates in the first and second locating units.

A fourth aspect of the present invention is a total hip replacement supporting system is one where the image-taking means holder and the drilling means holder in the total hip replacement supporting jig are integrated together. Then, the integrated holder holds the image-taking means or the medical drilling means in a mutually exchangeable manner according to need.

### Effects of the Invention

According to the total hip replacement arthroplasty supporting jig of the first aspect of the present invention, an existing image-taking means and an existing medical drilling means which are generally employed in an operating room are fitted to and held to the total hip replacement arthroplasty supporting jig that is so compact and so simplified as to be able to be fitted to the patient's acetabulum and thereby a precise form of the present patient's acetabulum can be grasped with ease and certainty by the image-taking means and besides a drilling position and drilling direction of the medical drilling means can be determined using each of two-dimensional coordinates. Hence, without the use of the conventional expensive and dedicated specific equipment, the patient's acetabulum can be formed into a precisely ideal form to thus permit an acetabular cup to be more easily located at an ideal locating position of the patient. Further, the equipment cost can be reduced by an amount resulting from eliminating the use of the expensive and dedicated special equipment.

Further, according to the total hip replacement arthroplasty supporting jig of the second aspect and the total hip replacement arthroplasty supporting system of fourth aspects of the present invention, the image-taking means and the medical drilling means need not be provided separately from each other to reduce the number of parts of the means, thus permitting the whole of the jig and system to be simplified.

Furthermore, according to the total hip replacement arthroplasty supporting system of the third aspect of the present invention, with reference to each two-dimensional coordinate specified in the total hip replacement arthroplasty supporting equipment, the drilling means holder in the total hip replacement arthroplasty supporting jig is regulated to allow a drilling position and a drilling direction to be determined. Hence, without the use of the expensive and dedicated special equipment being conventionally used, the patient's acetabulum can be precisely shaped into an ideal form to thus permit an acetabular cap to be more easily located an ideal locating position of the patient. Further, the equipment cost can be reduced by an amount resulting from eliminating the use of the expensive and dedicated special equipment.

### BRIEF DESCRIPTION OF THE DRAWING

FIG. 1 is a schematic diagram illustrating an overall structure of a total hip replacement arthroplasty supporting system.

FIG. 2 is a perspective view illustrating a first cylinder, a first leg, a second leg, a third leg and a first locating unit.

FIG. 3 is a perspective view illustrating overall structures of the first and second locating units.

FIG. 4 is a perspective view illustrating an overall structure of a second cylinder.

FIG. 5 is a block diagram illustrating a circuit configuration of a personal computer.

FIG. 6 is a perspective view illustrating a condition where a total hip replacement arthroplasty supporting jig is fitted to a patient.

### BEST MODE FOR CARRYING OUT THE INVENTION

The following is a detailed description of an embodiment of the present invention with reference to the appended drawings.

### (1) Overall Structure of the Total Hip Replacement Arthroplasty Supporting System

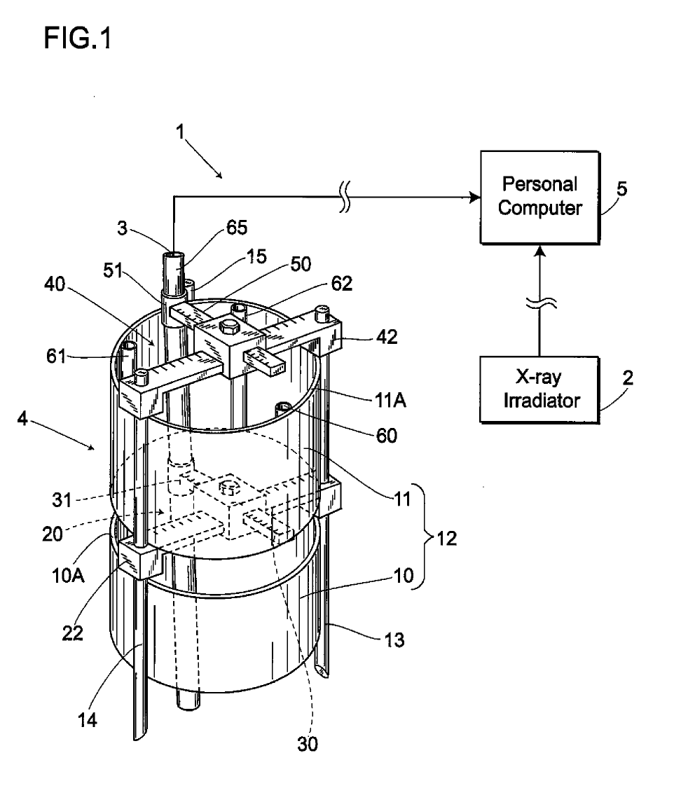

First of all, a gross outline of a total hip replacement arthroplasty supporting system is described below. In FIG. 1, reference numeral 1 denotes the total hip replacement arthroplasty supporting system according to the present invention as a whole. The total hip replacement arthroplasty supporting system comprises an X-ray irradiator 2 which obtains a hip joint X-ray photogram taken from two directions by irradiating an X-ray on a hip joint of a patient prior to applying the total hip replacement arthroplasty, the total hip replacement arthroplasty supporting jig 4 which is fitted directly to a patient's acetabulum exposed by dislocating a femoral head during surgery using the total hip replacement arthroplasty to hold an arthroscope (not shown) or a medical drill (not shown), and a personal computer 5 which can be connected electrically to the X-ray irradiator 2 and the arthroscope 3 to act as a total hip replacement arthroplasty supporting equipment. The personal computer 5 executes various processes based on an actual image data obtained from the X-ray photogram and the arthroscope 3, thereby providing information helpful for the total hip replacement arthroplasty to a surgeon engaged in surgery.

In actuality, the total hip replacement arthroplasty supporting system 1 sends out, e.g., patient's hip joint X-ray image data, taken from the two directions, which has been preliminarily obtained by the X-ray irradiator 2 prior to performing the surgery using the total hip replacement arthroplasty. Thus, given image processing is executed by the personal computer 5 based on the plurality of the X-ray image data and then pre-op bone image data can be produced by converting forms of a femur, acetabulum, pelvis around these femur into pre-op three-dimensional image data.

Then, using the personal computer 5, an operator (i.e., surgeons and supporting staffs who perform the total hip replacement arthroplasty) creates postoperative acetabular image data at the time of locating an acetabular cup ideally on the pelvis by drilling the patient's acetabulum using the total hip replacement arthroplasty or by performing other processing. Then, the postoperative acetabular image data are preliminarily stored for each patient before the surgery using the total hip replacement arthroplasty. Afterward, in the total hip replacement arthroplasty supporting system 1, a surgeon performs for a patient the surgery using the total hip replacement arthroplasty.

The total hip replacement arthroplasty supporting jig 4 is schemed so as to be fitted to the patient's acetabulum exposed during the surgery using the total hip replacement arthroplasty with the arthroscope 3 fitted and held thereto. As a result, the arthroscope 3 can directly take an image of the patient's acetabulum to send out the actual image data obtained at that time to the personal computer 5.

Then, the personal computer 5 executes a given arithmetic processes to the actual image data taken by the arthroscope 3 during the surgery using the total hip replacement arthroplasty. As a result, the personal computer 5 makes up stereophonic three-dimensional image data (hereunder, referred to as an acetabular three-dimensional actual image) of the present acetabulum of the patient who is undergoing the surgery using the total hip replacement arthroplasty, thus synthesizing the acetabular three-dimensional actual image and the postoperative acetabular image data.

Further, the personal computer 5 executes given arithmetic processes and then displays a postoperative acetabular image based on the postoperative acetabular image data on a given coordinate value beforehand set in the total hip replacement arthroplasty supporting jig 4 to present the result to the operators.

Then, in the total hip replacement arthroplasty supporting jig 4, the medical drill is fitted and held in substitute for the arthroscope during the surgery using the total hip replacement arthroplasty and besides determines a drilling position and drilling angle of the medical drill based on the coordinate presented from the personal computer 5 to drill the patient's acetabulum by the medical drill. Thus, an acetabular cup can be formed in an ideal acetabular form easy to locate.

### (2) Structure of the Total Hip Replacement Arthroplasty Supporting Jig

Next is a description of the total hip replacement arthroplasty supporting jig 4. This total hip replacement arthroplasty supporting jig has a size and weight which enable the jig to be directly fitted to the patient's acetabulum with the arthroscope 3 or the medical drill fitted and held thereto.

Virtually, the total hip replacement arthroplasty supporting jig 4 includes a jig main body 12 comprising an substantially cylindrical first cylinder 10 and a second cylinder 11 which is made separately from the first cylinder 10 and has the same form as that of the first cylinder 10 and a first, second and third legs 13, 14 and 15. By driving these first, second and third legs in an acetabulum, the jig main body 12 is arranged in the acetabulum in a standing manner.

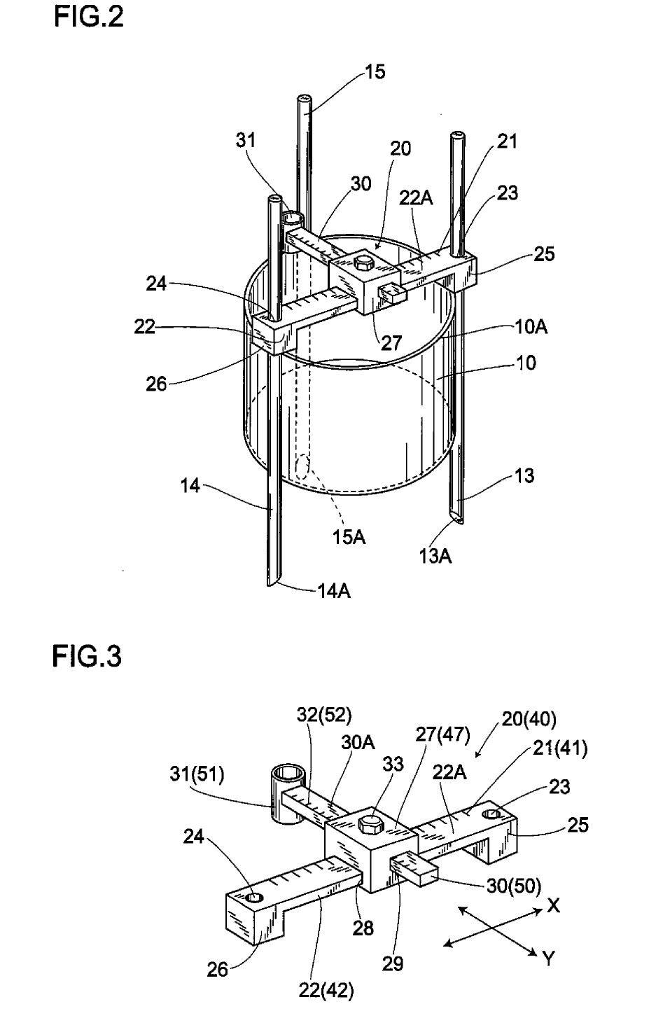

As shown in FIG. 2, virtually, the first, second and third legs 13, 14 and 15 which have all the same form and are baculiform are fixed firmly to an outer circumferential surface of the first cylinder 10 at regular intervals with longitudinal directions of the legs paralleled with one another. Pointed apical ends 13A, 14A and 15A of the first, second and third legs 13, 14 and 15 protrude from a lower end of the first cylinder.

In the present embodiment, since an acetabulum of an adult is generally about 40 mm to about 45 mm in diameter, the first cylinder 10 and the second cylinder 11 are selected as about 36 mm in outer diameter corresponding to the acetabulum of an adult and besides direct distances resulting from connecting by straight lines between adjacent first and second legs 13. 14 and between adjacent second and third legs 14, 15 are made up as about 40 mm.

The second cylinder 11 is inserted into a portion among the first, second and third legs 13, 14 and 15 with a first locating unit 20 made to intervene there between.

The first locating unit 20 includes, as shown in FIG. 3, a substantially-strip-shaped first coordinate unit 22 where scale marks 21 are inscribed on one surface 22A along a longitudinal direction (i.e., an x-axis direction in FIG. 3). Any two of the first, second and third legs 12, 14 and 15 can be inserted into through holes 23, 24 made in both ends of the first coordinate unit 22.

Both the ends of the first coordinate unit 22 are formed with protrusions 25, 26 protruding downward and the protrusions 25, 26 abut against the first cylinder 10. One surface 22A of the first locating unit can be substantially horizontally situated on an upper portion 10A of the first cylinder 10. In addition, in a central portion of the first coordinate unit 22, an substantially cubic coupling member 27 is provided in a freely slidable manner.

In reality, the first coordinate unit 22 is inserted into a through hole (hereunder, referred to as the through hole for the first coordinate unit) 28 made in a lower side of the coupling member 27. The coupling member 27 can side along the longitudinal direction (the x-axis direction in FIG. 3) of the first coordinate unit 22 and then abuts against protrusions 25, 26 at both the ends of the first coordinate unit 22 to thereby be prevented from disengaging from the first coordinate unit 22.

Additionally, the coupling member 27 is provided with a through hole (hereunder, referred to as the through hole for a second coordinate unit) 29 in an upper side thereof so as to be substantially orthogonal to the through hole 28 for the first coordinate. The second coordinate unit 30 is slidably inserted into the through hole 29 for the second coordinate unit.

The second coordinate unit 30 comprises a thin plate and is formed integrally with a cylindrical holding member 31 at one apical end thereof. Besides, scale marks 32 are inscribed along the longitudinal direction of one surface 30A. Accordingly, an indicating direction of the scale marks 32 becomes orthogonal substantially to an indicating direction (i.e., a y-axis direction in FIG. 3) of the scale marks 21 inscribed in the first coordinate unit 22.

The coupling member 27 is moved in given directions (the x- and y-axis directions) to thereby be fixed by an upper bolt 33 based on a first two-dimensional coordinate comprising the scale marks 21, 32 of the first and second coordinate units 22, 30, respectively, so that the holding member 31 is located at a specified position.

Besides, a second locating unit 40 with the same structure as that of the first locating unit 20 described above is arranged also in an upper portion 11A of the second cylinder 11 interposed among the first, second and third legs.

The second locating unit 40 comprises a first coordinate unit 42 inscribed with scale marks 41, a second coordinate unit 50 which is integrated with a holding member 51 at its apical end and is inscribed with scale marks 52, and a coupling member 47 which couples the first and second coordinate units 42, 50. As is the case with the first locating unit 20, the second locating unit 40 is arranged in the first, second, and third legs (FIG. 1).

In other words, in the case as shown in FIG. 1, when the first and second legs 13, 14 have been inserted into the through holes 23, 24 of the first locating unit 20, the first and second legs 13, 14 can be inserted into the through holes 23, 24 of the second locating unit 40.

A coupling member 47 is moved in given directions (the x- and y-axis directions) to be fixed at a specified position by an upper bolt 33 based on a second coordinate comprising the scale marks 41, 52 of the first and second coordinate units 42, 50, respectively, so that the holding member 51 is located at a specified position.

As a result, the holding member 31 in the first locating unit 20 and the holding member 51 in the second locating unit 40 are substantially aligned, so that the holding member 51 can be moved in the x- and y-axis directions on the upper portion 11A of the second cylinder 11.

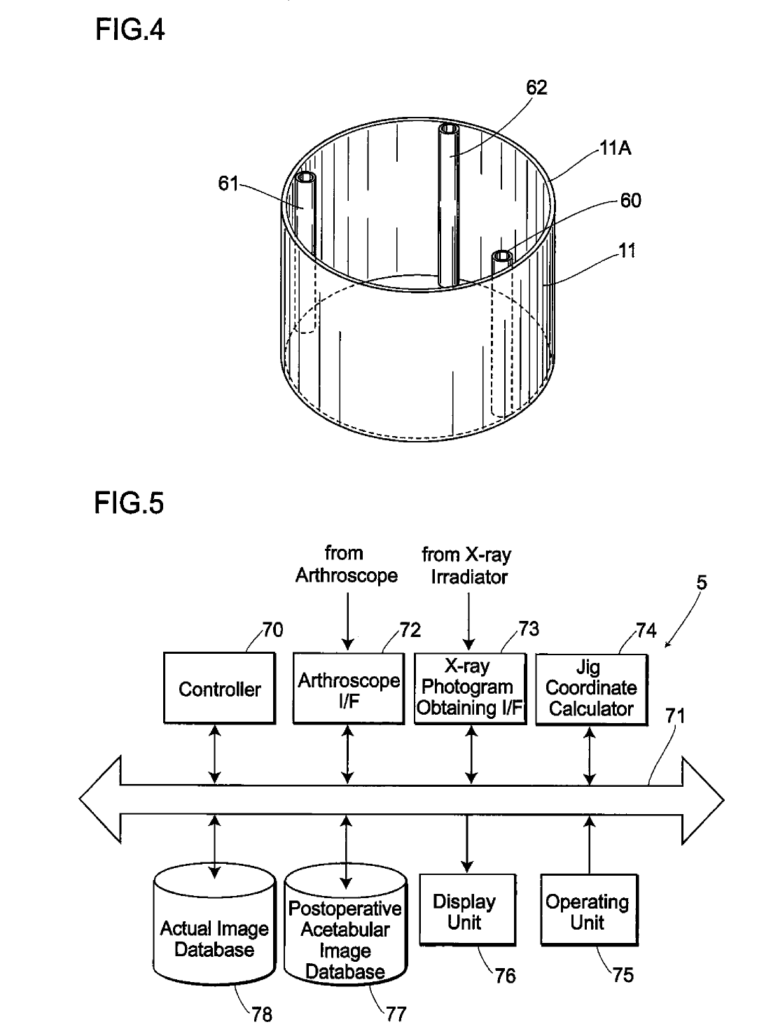

As shown in FIG. 4, a first luminous source pipe 60, a second luminous source pipe 61 and a third luminous source pipe 62 are fixed firmly to an inner circumferential surface of the second cylinder 11 at regular intervals with these pipes longitudinally paralleled with one another. A luminous source (not shown) such as LEDs or the like is mounted on each of the first, second and third luminous source pipes with the luminous sources directed to a side of the first cylinder 10. Thus, the luminous source illuminates the side of the first cylinder 10, illuming a region surrounded by the first cylinder 10.

A cylindrical pipe 65 can be inserted into and held to the holding members 31, 51 acting as an image-taking means holder and a drilling means holder. The arthroscope 3 or the medical drill is inserted into the pipe 65 and thereby the arthroscope 3 or the medical drill can be fitted to and held to a portion between the first and second locating units 20, 40.

In this case, the pipe 65, e.g., is held at an angle of about 30 degrees in relation to a central axis of the first and second cylinders 10, 11 by adjusting positions of the holding members 31, 51.

Accordingly, the arthroscope 3, acting as the image-taking means, is inserted into the pipe 65 from a top opening thereof and a camera lens of the arthroscope 3 is located in a bottom opening, so that a lower portion of the first cylinder 10 can be imaged by the arthroscope 3 from the angle of about 30 degrees in relation to the central axis of the first cylinder 10.

Then, data of actual images produced by imaging a central portion of the first cylinder 10 from about 30 degrees in relation to the central axis of the first cylinder 10 can be obtained at every 120 degrees by turning the arthroscope 3 120 degrees by 120 degrees. Then, the data of three-way-split actual image data are stored in the personal computer 5.

In addition to such a scheme, instead of the arthroscope 3, the medical drill is inserted into the first cylinder 10 from the top opening of the pipe 65 to allow the apical end of the medical drill to be exposed from the bottom opening of the side of the first cylinder 10, thus locating the apical end of the medical drill. Then, the holding members 31, 51 are moved and thereby the medical drill can be adjusted to be located at a given position and at a given angle.

### (3) Configuration of the Personal Computer

Next is a description of a configuration of the personal computer 5 with a reference to FIG. 5. The personal computer 5 includes a controller 70 configured as a microcomputer comprising a CPU (Central Processing Unit) (not shown), ROMs (Read Only memory) (not shown), RAMs (Random Access Memory) (not shown), hard disc drives and so on. Via a bus 71 connected to the controller 70 are an interface 72 of the arthroscope 3 acting as the image-taking means, an X-ray photogram obtaining interface 73, a jig coordinate calculator 74, an operating unit comprising a keyboard equipped with a plurality of operating keys and a mouth or the like, a display unit 76 such as a liquid display or the like, a postoperative acetabular image data base 77 acting as a storage means, and an actual image data base 78 which stores actual image data output from the arthroscope 3.

In this case, in the hard disc drives, in addition to a base program, installed are a three-dimensional X-ray photogram creating program, a postoperative acetabular image creating program, a three-dimensional actual image creating program, an image matching program, a jig coordinate calculating program or the like. The controller 70 retrieves accordingly various types of the programs installed in the hard disc drives to develop the programs retrieved in the RAMs and thereby controls various types of circuits of the personal computer 5 to execute various processing.

In this case, the controller 70, e.g., obtains, as an X-ray photogram, from the X-ray irradiator 2 via the X-ray photogram obtaining interface 73, a patient's acetabular X-ray photogram which has been obtained by the X-ray irradiator 2 and has been taken from two directions.

Depending on the three-dimensional X-ray photogram creating program, the controller 70 performs a given arithmetic process using relevant information obtained from the X-ray photogram data and thereby creates a three-dimensional image where a form of a patient's hip joint (a femur, a pelvis and an acetabulum) is realized in a three-dimensional fashion to create the three-dimensional image. Then, the controller 70 stores the three-dimensional image in the postoperative acetabular image data base 77 as three-dimensional acetabular data.

Afterward, the controller 70 executes the postoperative acetabular image creating program based on operating instructions input by the operator via the operating unit 75 to retrieve the three-dimensional acetabular image data stored in the postoperative acetabular image data base 77, displaying, on the display unit 76, a three-dimensional acetabular image based on the three-dimensional acetabular data.

Subsequently, in response to operating instructions given by the operator via the operating unit 75, the controller 70 displays on the display unit 76 the artificial acetabular image to be intended to be fitted to the patient together with the three-dimensional acetabular image. Then, e.g., based on image expanding and contracting instructions or the like via a cursor displayed on the display unit 76, the controller 70 expands, contracts, moves or rotates the three-dimensional acetabular image to deform the three-dimensional acetabular image.

Thus, the three-dimensional acetabular image is modified by the operator into an anatomically ideal acetabular form (hereunder, referred to as an ideal acetabular form) for fitting the acetabular cup to be treated as a postoperative image representing a state where the artificial acetabulum is fitted to the ideal acetabular form modified.

Then, as the postoperative acetabular image, the controller 70 stores, in the postoperative acetabular image data base 77, the postoperative image created by the operator via the operating unit 75.

Thereafter, the controller 70, acting as an acetabular three-dimensional actual image data creating means, obtains sequentially the actual image data via the arthroscope interface 72 from the arthroscope 3 that is fitted to and held to the total hip replacement arthroplasty supporting jig 4 during the surgery using the total hip replacement arthroplasty.

According to the three-dimensional actual image creating program, the controller 70 creates the acetabular three-dimensional actual image data based on the plurality of the actual image data obtained from the arthroscope 3 to display the acetabular three-dimensional image based on the acetabular three-dimensional actual image data on the display unit 76.

Next, according to the image matching program, the controller 70 executes an image matching process and thereby retrieves the postoperative acetabular image data from the postoperative acetabular image data base to display, on the display unit 76, the postoperative acetabular image based on the postoperative acetabular image data as well as the acetabular three-dimensional actual image.

Subsequently, the controller 70 moves and rotates the postoperative acetabular image and the acetabular three-dimensional actual image so that an edge line of the ideal acetabular form in the postoperative acetabular image and an edge line of the acetabular form in the acetabular three-dimensional image coincide with each other, thus performing various image synthesizing processes.

As a result, the controller 70 can associate the coordinate system of the postoperative acetabular image with the coordinate system of the acetabular three-dimensional image.

Then, the controller 70 produces a central axis line connecting a central point of the artificial acetabular image in the postoperative acetabular image and a central point of the ideal acetabular form in the postoperative acetabular image to specify, in a jig coordinate calculator 74, an intersecting point where the central axis line intersects with the first and second locating units 40 in the acetabular three-dimensional image.

In other words, the jig coordinate calculator 74, acting as a calculating means, calculates a value of the scale mark 21 at the intersecting point where the central axis line intersects with the first coordinate unit 22 in the first locating unit 20 and besides calculates a value of the scale mark 32 where the central axis line intersects with the second coordinate unit 30 in the first locating unit 20 to specify a first two-dimensional coordinate comprising these scale marks 21, 32 in the first locating unit 20.

Further, the jig coordinate calculator 74 calculates a value of the scale mark 41 at the intersecting point where the central axis line intersects with the first coordinate unit 42 in the second locating unit 40 and besides calculates a value of the scale mark 52 where the central axis line intersects with the second coordinate unit 50 in the second locating unit 40 to specify a second two-dimensional coordinate comprising these scale marks 41, 52 in the second locating unit 40.

Thus, for the postoperative acetabular image, the controller 70 can associate the first two-dimensional coordinate and the second two-dimensional coordinate with each other in the total hip replacement arthroplasty supporting jig 4.

### (4) Operation and Effect

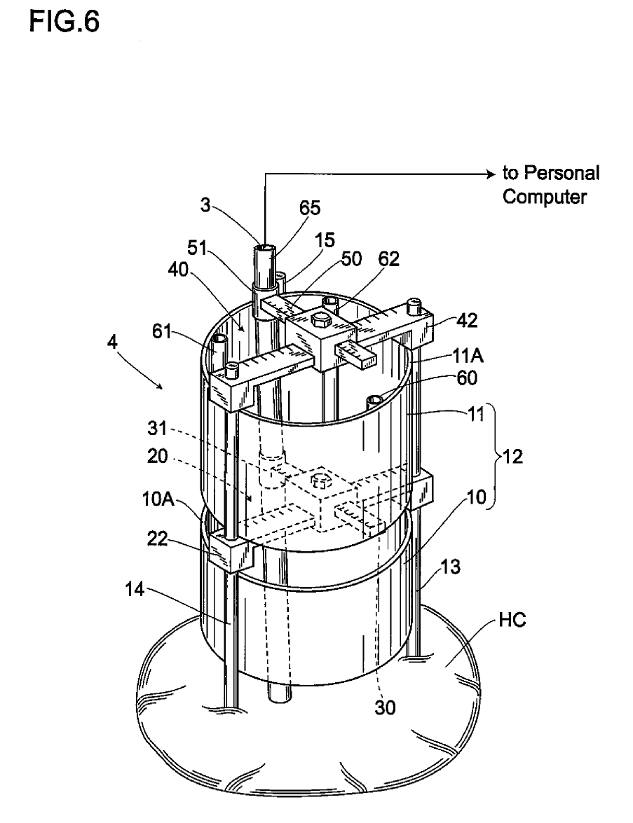

In the makeup described above, as shown in FIG. 6, a surgeon who performs the total hip replacement arthroplasty drives each of pointed apical ends 13A, 14A and 15A of the first, second and third legs, respectively, of the total hip replacement arthroplasty supporting jig 4 in an acetabulum HC exposed by dislocating a patient's femoral head during the surgery to fit directly the total hip replacement arthroplasty supporting jig 4 to the acetabulum HC.

In this case, in the total hip replacement arthroplasty supporting jig 4, the mutually orthogonal scale marks 21, 32 are provided in the first locating unit 20 and besides the mutually orthogonal scale marks 41, 52 are provided in the second locating unit 40 and thereby a given position on an opposite plane substantially opposed to the acetabulum HC can be represented by the first and second two-dimensional coordinates.

At this time, in the total hip replacement arthroplasty supporting jig 4, the first and second locating units 20, 40 are adjusted so that the pipe 65 makes an angle of about 30 degrees with a central axis of the first cylinder 10. By inserting the arthroscope 3 connected with the personal computer 5 into the pipe 65, an entire patient's acetabulum HC can be surely imaged from the direction of the angle of about 30 degree.

Further, in the total hip replacement arthroplasty supporting jig 4, the arthroscope 3 can be held with the arthroscope 3 fitted directly to the patient's acetabulum HC and hence the surgeon need not directly hold the arthroscope 3 to allow an adverse effect caused by a hand shake to be prevented without fail, thus permitting data of an actual image whose edge line of the patient's acetabulum HC has been clearly taken to be easily and surely obtained.

Furthermore, the surgeon rotates the arthroscope 3 by 120 degrees to image an edge portion of the acetabulum HC from the three directions. The actual image data thus obtained by the arthroscope 3 from the three directions are sent out in sequence to the personal computer 5 to be stored in the actual image database 78 of the personal computer 5.

Moreover, in the total hip replacement arthroplasty supporting jig 4, there are provided the first, second and third optical source pipes 60, 61 and 62 in the second cylinder 11 to make each LED emit light, illuminating the patient's acetabulum HC. Hence, even if the patient's acetabulum HC exists in a dark deep recess in the body, poor exposure of the arthroscope 3 can be prevented to allow the acetabulum HC to be certainly imaged with appropriate exposure.

On the one hand, when the operator gives prescribed operational instructions via the operating unit 75, in response to the instructions, the personal computer 5 retrieves the actual image data that had been output from the arthroscope 3 to be stored in the actual image data base 78 and then performs given arithmetic operations to thereby create the acetabular three-dimensional image, displaying the acetabular three-dimensional image on the display unit 76.

Moreover, the personal computer 5 creates a composite image where an edge line of the ideal acetabular form in the postoperative acetabular image preliminarily stored in the postoperative image data base 77 and an edge line of the acetabular form in the acetabular three-dimensional image have been made to coincide with each other to display the composite image on the display unit 76. Consequently, during the surgery using the total hip replacement arthroplasty, the surgeon can instantaneously compare the postoperative ideal acetabular form with patient's present acetabulum HC form, thus enabling an acetabulum HC region to be drilled by the medical drill to be visually recognized with ease.

Further, the personal computer 5 produces a central axis line connecting a central point of the artificial acetabular image in the postoperative acetabular image and a central point of the ideal acetabular form in the postoperative acetabular image. By representing, with the first two-dimensional coordinate and the second two-dimensional coordinate, an intersecting point where the central axis line intersects with the first locating unit 20 and the second locating unit 40, a drilling position and a drilling direction where the patient's acetabulum HC is drilled can be determined on the basis of the total hip replacement arthroplasty supporting jig 4.

After that, the surgeon being engaged in the total hip replacement arthroplasty extracts the arthroscope 3 from the pipe 65 of the total hip replacement arthroplasty supporting jig 4 and then inserts the medical drill into the pipe 65 instead of the arthroscope 3 to fit and hold the medical drill thereto. Then, based on various pieces of coordinate information presented by the personal computer 5 and the first and second two-dimensional coordinates in the total hip replacement arthroplasty supporting jig 4, the surgeon determines a drilling position and drilling direction of the medical drill, acting as a medical drilling means, thus continuing to drill the patient's acetabulum HC with accuracy and ease.

In this manner, in the total hip replacement arthroplasty supporting jig 4, by using the arthroscope 3 and the medical drill which have been conventionally typically used in an operating room, the personal computer 5, and the total hip replacement arthroplasty supporting jig 4 which can be fitted to the patient's acetabulum HC, the equipment cost can be even more reduced to less than that conventionally incurred due to no necessity of separate dedicated special equipment of large size.

Incidentally, the total hip replacement arthroplasty supporting jig 4 is detached after forming the patient's acetabulum HC by the medical drill. Afterward, the surgeon fits the acetabular cup to the patient's acetabulum, thus performing a usual total hip replacement arthroplasty.

According to the foregoing scheme, a jig main body 12 is fitted to the acetabulum HC via the first, second and third legs 13, 14 and 15. Further, the arthroscope 3 or the medical drill is fitted to and held to the first locating unit 20 whose surface substantially opposed to the acetabulum HC is indicated by the first two-dimensional coordinate and the second locating unit 40 whose surface substantially opposed to the acetabulum HC at a position distant further from the acetabulum HC than the locating unit 20 is indicated by the second two-dimensional coordinate.

In this manner, an existing arthroscope 3 and an existing medical drill are fitted to and held to the total hip replacement arthroplasty supporting jig 4 which is so compact and is so simplified as to be able to be fitted to the patient's acetabulum HC and thereby a precise form of the present patient's acetabulum HC can be easily and certainly grasped by the arthroscope 3. Besides, the drilling position and direction of the medical drill can be determined using the first two-dimensional coordinate and the second two-dimensional coordinate. Hence, the patient's acetabulum HC can be formed in a precisely ideal form without using such expensive and dedicated special equipment as has been conventionally used, thus enabling the acetabular cup to be located at the patient's ideal locating position with further ease. Besides, the expensive and dedicated special equipment becomes unnecessary, leading to a reduction in equipment cost by just that much.

Further, the arthroscope 3 and the medical drill are fitted to and held to the total hip replacement arthroplasty supporting jig 4 via one set of the holding members 31, 51. Hence, there is no need to provide holding means separately for both the arthroscope and the medical drill to permit the number of parts to be reduced, thus allowing a simple structure to be provided as a whole.

Furthermore, in the total hip replacement arthroplasty supporting system 1, the existing arthroscope typically used in an operating room is fitted to and held to the total hip replacement arthroplasty supporting jig 4 which is so compact and so simplified as to be able to be fitted to the patient's acetabulum. Then, the precise form of the present patient's acetabulum HC is imaged by the arthroscope 3 to be sent out to the personal computer 5 as an actual image data. Based on the actual image data sent out, a given position of the ideal acetabular form in the postoperative acetabular image data is specified by the personal computer 5 using the first and second two-dimensional coordinates in the first and second locating units 20, 40.

Accordingly, with reference to the first and second two-dimensional coordinates which are specified by the personal computer 5 and are in the first and second locating units 20, 40, the holding members 31, 51 in the total hip replacement arthroplasty supporting jig 4 are adjusted to permit the drilling position and drilling direction of the medical drill to be determined. Hence, the patient's acetabulum HC can be formed in a precisely ideal form without the use of such expensive and dedicated special equipment as has been conventionally used, thus enabling the acetabular cup to be located on the patient's ideal position with further ease. Further, the general personal computer is used and thereby the expensive and dedicated special equipment becomes unnecessary, thus permitting a reduction in equipment cost by just that much.

In addition, the present invention is not limited to the forging embodiment and various modifications are possible within the scope of the subjects described in the claims of the present invention. For example, various types of CCDs may be employed as an image-taking means and a medical reamer as a medical drilling means.

In the embodiment described above, it has been described that the arthroscope 31 and the medical drill 51 are held via the same holding members 31, 51. The present invention, however, is not limited to the structure and the arthroscope 31 and the medical drill 51 may be held via different exclusive holding members.

1. A total hip replacement arthroplasty supporting jig comprising:
legs which allows a jig main body to be fitted to a patient's acetabulum exposed by dislocating a femoral head during surgery using a total hip replacement arthroplasty,
a first locating unit provided in said jig main body to indicate, using a two-dimensional coordinate, an opposite plane substantially opposed to said patient's acetabulum,
a second locating unit provided in said jig main body to indicate, using said two-dimensional coordinate, an opposite plane substantially opposed to said acetabulum at a position further distant from said patient's acetabulum than is said first locating unit,
an image-taking means holder provided in a freely slidable manner in said first and second locating units to hold an image-taking means for imaging said patient's acetabulum, and
a drilling means holder provided in a freely slidable manner in said first and second locating units to hold a medical drilling means for drilling said patient's acetabulum.

2. A total hip replacement arthroplasty supporting jig according to claim 1, wherein said image-taking means holder and said medical drilling means holder are integrated with each other to hold said medical drilling means or said image-taking means in a mutually exchangeable manner according to need.

3. A total hip replacement arthroplasty supporting system comprising:
a total hip replacement arthroplasty supporting equipment and
a total hip replacement arthroplasty supporting jig,
wherein said total hip replacement arthroplasty supporting jig comprises:
legs which allow a jig main body to be fitted to a patient's acetabulum exposed by dislocating a femoral head during surgery using a total hip replacement arthroplasty,
a first locating unit provided in said jig main body to indicate, using a two-dimensional coordinate, an opposite plane substantially opposed to said patient's acetabulum,
a second locating unit provided in a jig main body to indicate, using said two-dimensional coordinate, an opposite plane substantially opposed to said patient's acetabulum at a position further distant from said acetabulum than said first locating unit,
an image-taking means holder provided in a freely slidable manner in said first and second locating units to hold an image-taking means for imaging said patient's acetabulum,
a drilling means holder provided in a freely slidable manner in said first and second locating units to hold a medical drilling means for drilling said patient's acetabulum, and
wherein said total hip replacement arthroplasty supporting equipment comprises:
a storage means which stores a postoperative acetabular image data created by preliminarily converting a postoperative ideal patient's acetabular form into a three-dimensional image before surgery using said total hip replacement arthroplasty,
an image obtaining means which obtains actual image data of said patient's acetabulum imaged by said image-taking means,
an acetabular three-dimensional actual image data creating means which converts said actual image data into three-dimensional image data to create acetabular three-dimensional actual image data associated with each of two-dimensional coordinates in said first and second locating units, and
a calculating means which makes said acetabular three-dimensional actual image data and said postoperative acetabular image data retrieved from said storage mean coincide with each other to specify a given position of said ideal acetabular form in said postoperative acetabular image data using each of said two-dimensional coordinates in said first and second locating units.

4. A total hip replacement arthroplasty supporting system according to claim 3, wherein said image-taking device holder and said drilling device holder in said total hip replacement arthroplasty supporting jig are integrated with each other to hold said image-taking device or said medical drilling means in a mutually exchangeable manner.
